(19) United States
(12) Patent Application Publication
(10) Pub. No.: US 2017/0020384 A1
(43) Pub. Date: Jan. 26, 2017

(54) Laryngoscope

(71) Applicant: Opticks Inc., Mendota Heights, MN (US)

(72) Inventors: Adam Fitzgerald, Minneapolis, MN (US); Sara Gergen, Mendota Heights, MN (US); Anders Hanson, Los Angeles, CA (US); Kari Roberts, St. Paul, MN (US); Robert Shane Robins, Mendota Heights, MN (US); Babak Tabesh, Minneapolis, MN (US)

(73) Assignee: Opticks Inc.

(21) Appl. No.: 15/217,950

(22) Filed: Jul. 22, 2016

Related U.S. Application Data
(60) Provisional application No. 62/195,460, filed on Jul. 22, 2015.

(51) Int. Cl. A61B 1/267; A61B 1/06; A61B 1/07; A61B 1/00

(52) U.S. Cl. CPC A61B 1/267; A61B 1/0008; A61B 1/0684; A61B 1/07

(57) ABSTRACT

A laryngoscope includes a blade having a distal end and a proximal end, wherein a blade includes a curved portion adjacent the distal end, a housing within the blade adjacent the curved portion, a light source positioned within the housing, and a handle adjacent the proximal end.

### CROSS-REFERENCE TO RELATED APPLICATIONS

This application incorporates by reference and claims the benefit of priority to U.S. Provisional Application No. 62/195,460 filed on Jul. 22, 2015.

### BACKGROUND OF THE INVENTION

The present invention relates to laryngoscopes that provide sufficient lighting to a patient's vocal cords and glottis. Specifically, the present invention provides a laryngoscope having a light source disposed within a transparent or translucent plastic housing near the distal end of the blade.

Laryngoscopy is a procedure used to view the vocal cords and glottis of a patient. The procedure is performed using a laryngoscope, and allows an endotracheal tube to be placed in the trachea of the patient to assist in ventilation and oxygenation. The placement of the endotracheal tube is commonly called intubation. Intubation involves using a laryngoscope in the patient's oral cavity to obtain direct visualization of the vocal cords and glottis, inserting an endotracheal tube through the vocal cords into the trachea, removing the laryngoscope, and initiating positive pressure ventilation through the endotracheal tube.

Intubation is often necessary when a patient is experiencing respiratory distress requiring mechanical ventilation. Problems with intubation can lead to hypoxia, where the patient is deprived of an adequate oxygen supply, and/or hypercarbia, where carbon dioxide produced by the patient is inadequately expelled and therefore is present in the patient at abnormally elevated levels. Intubation is often associated with stress and adverse physiologic effect, such as a decreased heart rate, fluctuations in blood pressure, decreased oxygen levels, and increased blood pressure to the brain. Some patients have additional complications.

The design of the laryngoscope has changed very little over the last several decades. A conventional laryngoscope is composed of a metal handle with a detachable blade that includes a light source extending therefrom. During the procedure, the blade is inserted into the patient's mouth. The tongue is lifted to view the larynx and gain access to the vocal cords and glottis. Upon completion of the procedure, the laryngoscope is cleaned or sterilized using the manufacturer recommended procedures for later reuse.

In some designs, a light source is positioned on the blade to illuminate the patient's anatomy during the procedure. The light extends from a mid-section of the blade and is directed toward the vocal cords and glottis of the patient. The design of these instruments historically focused on adult patients. In neonatal patients, which can weigh as little as 350 grams, the mouth can be very small, and the location of the light source in relationship to the distal end of the blade is critical to the success of the intubation procedure. Proximity of the light to the distal end of the blade improves functionality dramatically. When conventional laryngoscopes are used in smaller neonates, the light may be completely outside of the mouth when the blade is inserted, making visualization of the vocal cords difficult. The majority of devices have placed the light too far away from the neonates' glottis and vocal cords.

Conventional laryngoscope designs use a focal light that illuminates only a portion of the oral cavity. The blade then needs to be maneuvered in the patient's mouth in order to locate the glottis and vocal cords so that the endotracheal tube can be successfully placed in the trachea. Each patient's anatomy is different and locating these important landmarks to ensure proper placement of the endotracheal tube can be difficult with only localized portions of the anatomy illuminated.

Additionally, some laryngoscopes targeted towards adults incorporate a video camera in close proximity to the anatomy and displays the anatomy on a screen. Such devices are expensive, and therefore not prevalent. There is typically one video device for multiple hospital units making the device unavailable in emergency situations. This is inconvenient for practitioners. Delaying the intubation due to inaccessible equipment may directly impact patient outcomes. Additionally, laryngoscopes with video capability are difficult to use, resulting in intubation taking longer than when using laryngoscopes without video capability. Video laryngoscopes also require sterilization between uses.

In order for these devices to be used in a variety of patients of different sizes, the blade of the laryngoscope comes in different styles and blade lengths. Even so, these devices only partially function in some patients. For example, neonates have unique anatomical characteristics. A neonate's weight can range from 350-5000 grams, and their oral cavity is only a few centimeters in diameter and length. The majority of neonatal laryngoscopes are conventional (i.e., adult) laryngoscopes with a blade length that is scaled down for use in the neonate. There are only three blades sizes currently available for neonates, and each utilizes the same size light bulb and blade width and depth, with only the length of the blade having been modified. Further, the light source extends outwardly from the blade and is therefore along the user's line of sight, particularly when used with neonates. With the smaller infants, a large portion of the user's field of view is blocked by the light source. Additionally, in smaller infants, the light source may not enter the oral cavity when the blade is fully inserted causing the light to not adequately illuminate the anatomy. In the larger infants, the tongue often obscures the field of vision because the blade itself is too small to hold the tongue safely out of the way.

Further, the intubation process may result in adverse effects if the procedure is not performed quickly enough. Infants are not able to receive positive pressure ventilation during the procedure and may rapidly decompensate if the duration of the procedure exceeds the lung reserve of oxygen. Infants are often premedicated prior to the procedure and the duration of each attempt at intubation is limited. The American Academy of Pediatrics Neonatal Resuscitation Program (AAP NRP) recommends that intubation attempts be limited to 30 seconds. Studies have shown that intubation attempts frequently exceed 30 seconds, with some attempts lasting up to several minutes. The duration of the procedure may be monitored by the user or an additional provider using a timer or clock.

Conventional devices are reusable and require sterilization after every use, which is time consuming and costly. It is also difficult to maintain device sterilization at the patient bedside. Video laryngoscopes are reusable and require sterilization. Further, the standard of care has recently evolved to using a sterilized device for every intubation. There are very few options for disposable laryngoscopes available. The devices that are designed as single use are preferred both from a cost, safety and ease of use perspective.

Accordingly, there is a need to provide a more versatile laryngoscope for improved visualization and illumination of the oral cavity and include a feature for warning the user that the insertion of the laryngoscope has reached a specified duration.

### SUMMARY OF THE INVENTION

The present application provides a laryngoscope that includes the light source positioned within a transparent or translucent housing within the blade of the device. The transparent or translucent housing disperses light into a broad illumination distribution within the oral cavity during use. By placing the light source within the blade, the user has an unobstructed view of the patient's vocal cords and glottis and a localized light source.

The blade extends from a handle that includes a timer feature adjacent to the blade. The timer includes indicator lights that the user can easily monitor during the procedure. First, second, and third indicator lights are embedded within the handle. In one embodiment, the first, second, and third indicator lights are green, yellow, and red, respectively. The laryngoscope is preprogrammed so that the indicator lights are illuminated at specific intervals during the procedure. The green light is illuminated once the start button is activated and remains illuminated during the first portion of the procedure. As the duration of the procedure approaches a preprogrammed end time, the yellow light is illuminated to warn the user. The red light is illuminated once the duration reaches the end time to inform the user that the procedure is going beyond the time frame recommended by the AAP NRP, and that the procedure should be completed or suspended.

In one embodiment, the end time corresponds to the time frame recommended by the AAP NRP, 30 seconds. In this embodiment, the yellow light turns on at 20 seconds and the red light turns on at 30 seconds after activating the start button.

An advantage of the present design is to provide sufficient lighting to the patient's vocal cords and glottis.

A further advantage of the present design is to provide an unobstructed view of the patient's vocal cords and glottis during endotracheal intubation.

Another advantage of the present design is adequate visualization and access to the vocal cords and glottis of neonatal patients.

An additional advantage of the present design is the convenient timing feature that informs the user that the procedure should be completed or suspended per recommendation of AAP NRP.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitation. In the figures, like reference numerals refer to the same or similar elements.

FIG. 1 is an isometric view of a blade of a laryngoscope in accordance with the present disclosure from the front.

FIG. 2 is an isometric view of the blade of FIG. 1 from of the back.

FIG. 3 is an isometric view of a handle of the laryngoscope of FIG. 1.

FIG. 4 is an isometric view of a timer of the handle of FIG. 3.

### DETAILED DESCRIPTION OF THE INVENTION

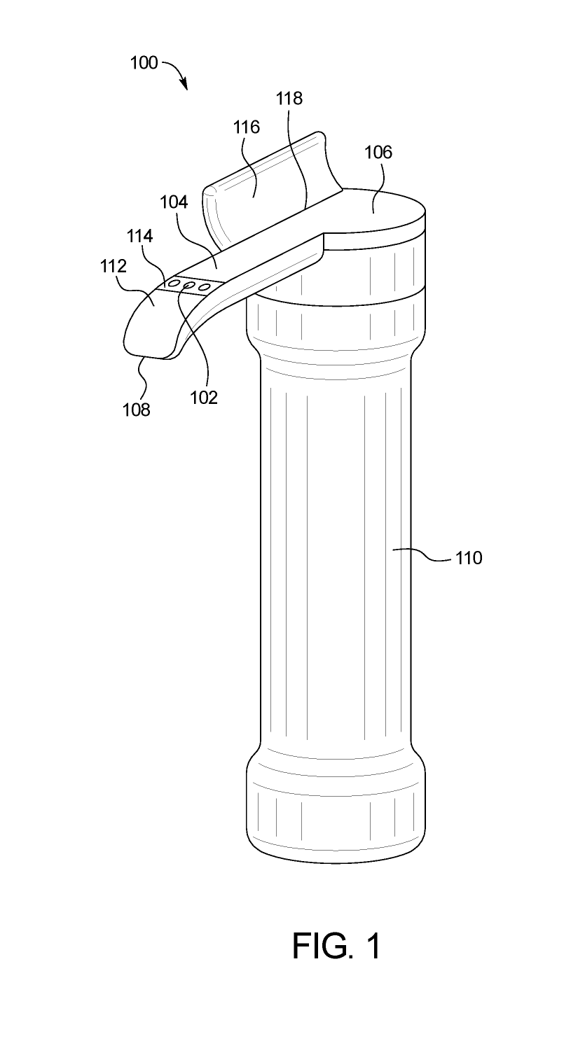

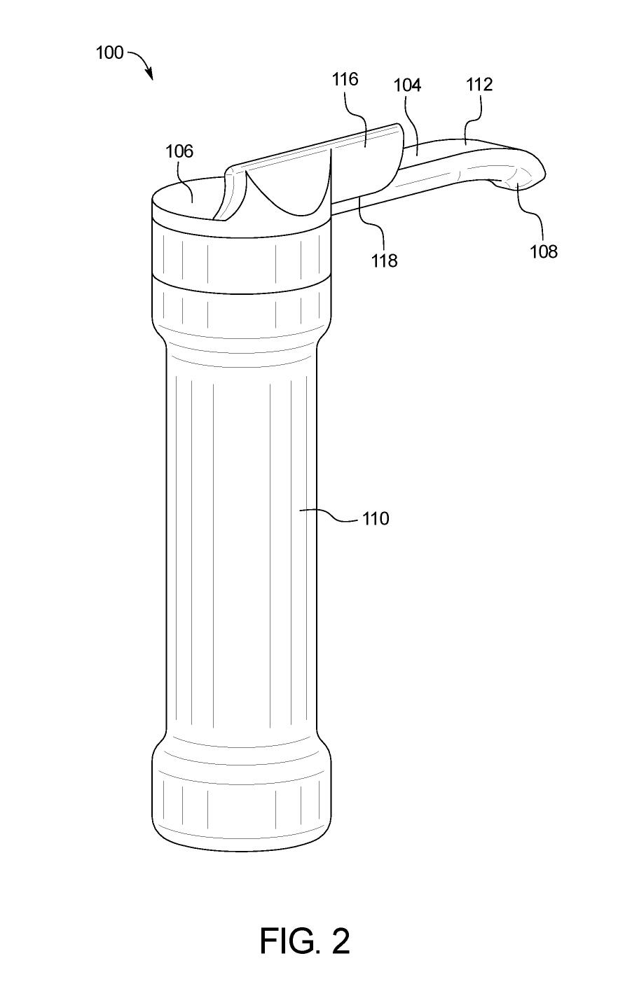

Referring to FIGS. 1 and 2, the present application discloses a laryngoscope 100 including a light source 102 housed within and extending across a width of a blade 104. More specifically, the blade 104 includes a proximal blade end 106 and a distal blade end 108 opposite the proximal blade end 106, with a handle 110 extending from the proximal blade end 106. The distal blade end 108 includes a curved portion 112 so that it is easily manipulated into the patient's oral cavity. The light source 102 is positioned within the blade 104 adjacent the curved portion 112. Specifically, the light source 102 is positioned within a housing 114 within the blade 104. A side wall 116 projects from a longitudinal side 118 of the blade 104 to further open the oral cavity during use. In some embodiments, the handle 110 includes a timer 120 immediately adjacent to the blade 104.

The light source 102 of the laryngoscope 100 is housed in a transparent or translucent housing 114 that causes light to be dispersed throughout the oral cavity. Embedding the light source 102 within the blade 104 reduces the amount of maneuvering required to locate the vocal cords and glottis. Additionally, in contrast to a light attachment to a conventional laryngoscope, the light source 102 embedded within the blade 104 the laryngoscope 100 of the present application prevents any obstruction of the view of the user. The housing 114 may be made of any material that allows light to pass through, such as transparent or translucent plastic.

A high intensity light source 102 such as a light-emitting diode (LED) or fiber optic may be used. The laryngoscope 100 may include one or more LEDs, fiber optics, or other sources. The light source 102 is selected and positioned within the laryngoscope 100 such that heat emitted from the light source 102 does not harm the patient.

The blade 104 of the laryngoscope 100 may also have a thickness such that it does not block the operators view. In some embodiments, a portion of the blade 104 that is pressed against the tongue to hold more of the tongue out of the field of view is comprised of a material having a low durometer reading. One example of a low durometer material is a soft rubber, although other materials may be used. The low durometer material improves stabilization of the tongue, which may contribute to the speed and ease at which the intubation procedure can be accomplished. The laryngoscope 100 of the present application minimizes the discomfort and probe-induced stress for the patient as well as may reduce the amount of time the patient does not have a secure airway and/or is with hypoxia and hypercarbia.

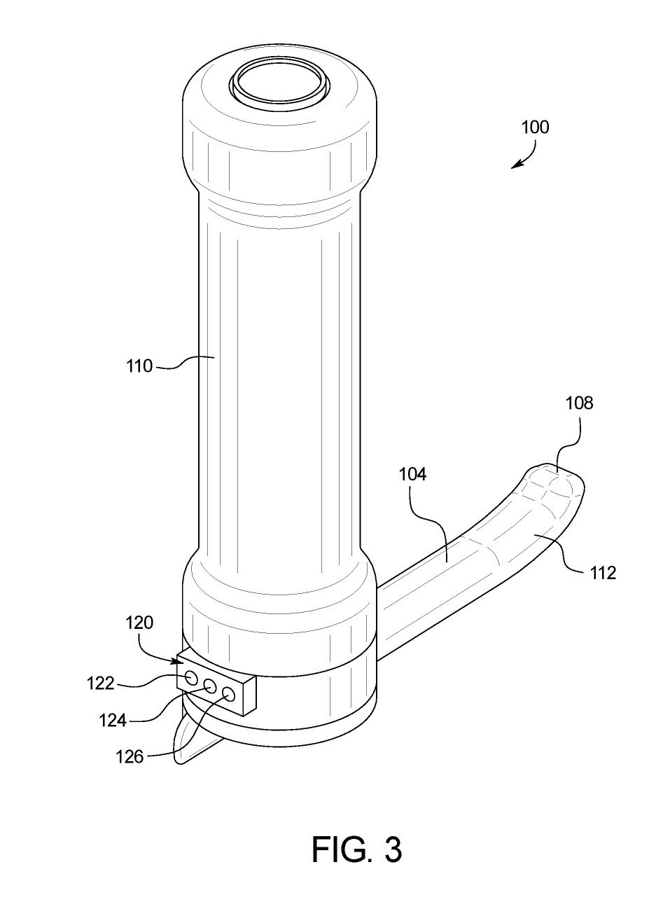

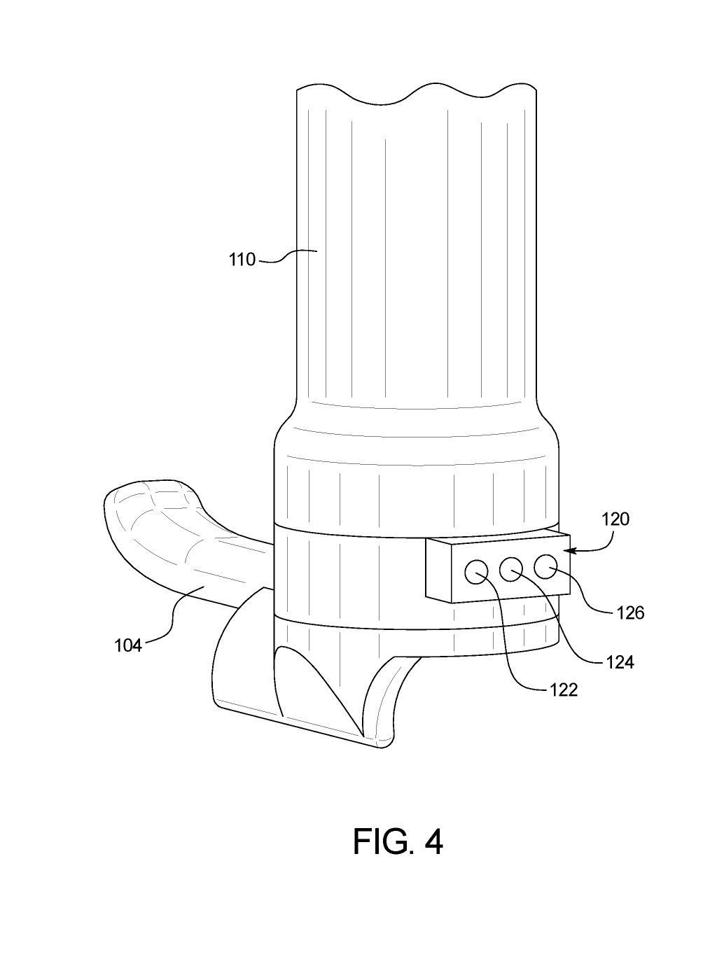

Referring to FIGS. 3 and 4, the timer 120 is located within the handle 110 immediately adjacent to the blade 104 so that the user can easily monitor the timer 120 during the procedure. In the illustrated embodiment, the timer 120 includes first, second, and third indicator lights 122, 124, 126 that are preprogrammed to be are illuminated at specific intervals during the procedure. The first light 122 is green and is illuminated once the start button is activated. The second light 124 is yellow, and is illuminated as the procedure approaches a preprogrammed end time. For example, the second light 124 may be illuminated about ten seconds before the end time. The third light 126 is red, and is illuminated once the duration reaches the end time to alert the user that the procedure should be completed or suspended.

In other embodiments, the indicator lights 122, 124, 126 may be different colors. In another embodiment, the timer 120 may include a single indicator light that flashes at different rates at different intervals during the procedure. For example, the single light may remain solid at the start of the duration, may start flashing at a first rate as the duration approaches the end time, and may start flashing at a second, faster rate when the duration reaches the end time.

In one embodiment, the end time corresponds to the time frame recommended by the AAP NRP, 30 seconds. In this embodiment, the yellow light turns on at 20 seconds and the red light turns on at 30 seconds after activating the start button. In some embodiments, the laryngoscope 100 may be preprogrammed with a specific duration and intervals for triggering the indicator lights 122, 124, 126. In other embodiments, the laryngoscope 100 may include functionality to allow the user to adjust the duration and intervals for triggering the lights 122, 124, 126.

During use, the blade 104 is inserted into the mouth of the patient with the low durometer material facing up and the light source 102 facing down while the patient is in the supine position. The blade 104 is then slightly pulled superior by the user to trap and move the tongue out of the line of sight. The blade 104 is then lifted with a superficial motion. This move enables the user to view of the glottis and the vocal cords.

The improvements of the laryngoscope 100 of the present application are applicable to all patient populations. For example, the laryngoscope 100 may be used with adult patients of various sizes as well as neonatal patients. In some embodiments, the blade may come in different sizes to accommodate the various patient populations.

The drawing figure depicts one or more implementations in accord with the present concepts, by way of example only, not by way of limitations. Additional objects, advantages and novel features of the examples will be set forth in part in the description above, and in part will become apparent to those skilled in the art upon examination of the following description and the accompanying drawing or may be learned by production or operation of the examples. The objects and advantages of the concepts may be realized and attained by means of the methodologies, instrumentalities and combinations as described.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A laryngoscope comprising:
a blade having a distal end and a proximal end, wherein a blade includes a curved portion adjacent the distal end;
a housing within the blade adjacent the curved portion;
a light source positioned within the housing; and
a handle adjacent the proximal end.

2. The laryngoscope of claim 1, wherein the housing comprises a transparent material.

3. The laryngoscope of claim 1, wherein the housing comprises a translucent material that spreads a light distribution of the light source.

4. The laryngoscope of claim 1, wherein the light source comprises a light-emitting diode.

5. The laryngoscope of claim 1, further including a timer programmed to be activated at a plurality of time intervals.

6. The laryngoscope of claim 5, wherein the timer includes first, second, and third indicators that correspond to first, second, and third time intervals.

7. The laryngoscope of claim 6, wherein the first, second, and third indicators comprise first, second, and third indicator lights.

8. The laryngoscope of claim 7, wherein the first indicator light is illuminated when the timer is activated.

9. The laryngoscope of claim 8, wherein the timer is programmed with an end time, and wherein the second indicator light is illuminated approximately ten seconds before the timer approaches the end time.

10. The laryngoscope of claim 9, wherein the third indicator light is illuminated when the timer reaches the end time.
